(19) United States
(12) Patent Application Publication
(10) Pub. No.: US 2026/0355399 A1
(43) Pub. Date: Nov. 12, 2026

(54) WEARABLE PERITONEAL DIALYSIS SYSTEM

(71) Applicants: BAXTER INTERNATIONAL INC., Deerfield, IL (US); BAXTER HEALTHCARE SA, Glattpark (Opfikon) (CH)

(72) Inventor: Narendar VENKATACHALAPATHY, Bangalore (IN)

(21) Appl. No.: 19/113,689

(22) PCT Filed: Oct. 4, 2023
(86) PCT No.: PCT/US2023/034446

§ 371 Date: Mar. 20, 2025

(30) Foreign Application Priority Data
Oct. 14, 2022 (IN) 202241058812

(51) Int. Cl. A61M 1/28; A61M 1/16

(52) U.S. Cl. CPC A61M 1/287; A61M 1/1601; A61M 2202/0413; A61M 2205/58

(57) ABSTRACT

A wearable peritoneal dialysis system includes a PD fluid supply assembly including an outer container, and an inner PD fluid supply container sealed within the outer container; a pumping unit including an air pump having a positive pressure outlet and a negative pressure outlet; a PD fluid drain assembly including an outer leg container configured to be worn around a patient's leg, an inner PD fluid drain container sealed within the outer leg container; and a control unit configured to cause (i) positive pneumatic pressure to be supplied from the positive pressure outlet to the outer container to force fresh PD fluid to flow from the inner PD fluid supply container to the patient and (ii) negative pneumatic pressure to be supplied from the negative pressure outlet to the inner container to force used PD fluid to flow from the patient towards the inner PD fluid drain container.

### PRIORITY CLAIM AND CROSS-REFERENCE TO RELATED APPLICATIONS

The present application is a national stage filing of PCT/US2023/034446, filed Oct. 4, 2023, which claims priority to Indian Patent App. No. 202241058812, filed Oct. 14, 2022, the entire contents of which are hereby incorporated by reference.

### BACKGROUND

Due to various causes, a person's renal system can fail. Renal failure produces several physiological derangements. For instance, it is no longer possible for a person with renal failure to balance water and minerals or to excrete daily metabolic load. Additionally, toxic end products of metabolism, such as, urea, creatinine, uric acid and others, may accumulate in a patient's blood and tissue.

Reduced kidney function and, above all, kidney failure is treated with dialysis. Dialysis removes waste, toxins and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across a semi-permeable dialyzer between the blood and an electrolyte solution, called dialysate or dialysis fluid, to cause diffusion. The diffusion occurs externally from the patient, where an extracorporeal circuit is used for removing uncleansed blood and returning cleansed blood to the patient.

Hemofiltration (“HF”) is an alternative renal replacement therapy that relies on a convective transport of toxins from a patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large toxic molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Another type of kidney failure therapy is peritoneal dialysis (“PD”), which infuses a dialysis solution, also called dialysis fluid, into a patient's peritoneal chamber via a catheter. The dialysis fluid is in contact with the peritoneal membrane in the patient's peritoneal chamber. Waste, toxins and accumulated water (ultrafiltration) pass from the patient's bloodstream, through the capillaries in the peritoneal membrane, and into the dialysis fluid due to diffusion and osmosis, i.e., an osmotic gradient occurs across the membrane. An osmotic agent in the PD dialysis fluid provides the osmotic gradient. Used or spent dialysis fluid is drained from the patient, removing waste, toxins and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis, and continuous flow peritoneal dialysis (“CFPD”). CAPD is a manual dialysis treatment. Here, the patient manually connects an implanted catheter to a drain to allow used or spent dialysis fluid to drain from the peritoneal chamber. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal chamber, where the transfer of waste, toxins and excess water takes place. After a dwell period, the patient repeats the manual dialysis procedure, for example, four times per day. Manual peritoneal dialysis requires a significant amount of time and effort from the patient, leaving ample room for improvement.

Automated peritoneal dialysis (“APD”) is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh dialysis fluid and to a fluid drain. APD machines pump fresh dialysis fluid from a dialysis fluid source, through the catheter and into the patient's peritoneal chamber. APD machines also allow for the dialysis fluid to dwell within the chamber and for the transfer of waste, toxins and excess water to take place. The source may include multiple liters of dialysis fluid including several solution bags.

APD machines pump used or spent dialysate from the patient's peritoneal cavity, though the catheter, and to the drain. As with the manual process, several drain, fill and dwell cycles occur during dialysis. A “last fill” may occur at the end of the APD treatment. The last fill fluid may remain in the peritoneal chamber of the patient until the start of the next treatment, or may be manually emptied at some point during the day.

In any of the above modalities using an automated machine, the automated machine is stationary, e.g., in a patient's home. The form factor for APD machines requires a patient to be immobilized in a bed or chair for the duration of a kidney failure therapy, which can last up to eight hours. As such, a patient undergoing an APD treatment is generally tied to the machine, which is why APD treatments typically occur at night. Certain patients would rather perform a PD treatment while they are awake, however, so that they may try to avoid alarms that in many cases are due to the patient's sleeping position and to be able to tend to alarms without being awakened. The patient's may then enjoy their sleep without having to integrate a PD treatment. Additionally, some patients who perform PD treatment at night while asleep still have to perform a midday PD fluid exchange, which may require the patient to have to return home where the supplies for performing the midday exchange are stored.

A need accordingly exists for a portable or mobile machine for peritoneal dialysis while the patient is moving or is in a situation requiring the ability to move.

### SUMMARY

Example systems, methods, and apparatus are disclosed herein for a wearable peritoneal dialysis (“PD”) system that performs a PD treatment while the patient is either moving or in a situation requiring the patient to have to move. The example PD system in one embodiment includes three primary components, namely, a PD fluid supply assembly, a pumping unit and a PD fluid drain assembly. The PD fluid supply assembly includes an outer supply container and an inner PD fluid supply container. The outer supply container may be rigid, such as a rigid plastic, or be a flexible container, such as a flexible airtight fabric, e.g., canvas, which does not stretch substantially when placed under positive pneumatic pressure. The outer supply container is in one embodiment openable, e.g., hinged open, so as to be able to receive a new inner PD fluid supply container, which may be a standard presterilized PD fluid bag. The outer supply container includes a sealed interface, such that when the outer supply container is closed about the inner PD fluid supply container, the outer supply container seals in an airtight manner about the inner PD fluid supply container. The outer supply container may include one or more latch that releasably closes to compress the sealed interface during operation of the wearable PD system of the present disclosure. If the outer supply container is flexible, e.g., canvas, the sealed interface may include an airtight zipper. Regardless of the material for the outer supply container, the sealed interface in an embodiment also seals around a supply connector, e.g., standard PD fluid bag connector, which extends from the inner PD fluid supply container. In this way, the supply connector may be connected to a PD fluid supply line outside of the outer supply container. In a similar manner, the outer supply container includes a pneumatic connector that may be accessed and connected to a positive pneumatic line outside of the outer supply container.

The outer supply container is reusable and in one embodiment includes one or more sensor located at a bottom of the container for sensing with the PD fluid located within the inner PD fluid supply container is almost complexly gone, having been delivered to the patient. The sensor may be any type of sensor that can detect, through a wall of the inner PD fluid supply container, whether PD fluid or air is present. Such a sensor may for example be an ultrasonic sensor, magnetic sensor, capacitive sensor or inductive sensor. The outer supply container and inner PD fluid supply container are here sized such that the inner PD fluid supply container is held snugly within the outer supply container so that the sensor is consistently pressed up against a wall of the inner fluid supply container to provide an accurate output signal.

The PD fluid supply assembly in one embodiment further includes a backpack worn by the patient and into which the outer supply container and the nested inner PD fluid supply container are placed while the patient moves from one location to another for whatever reason, and while the patient receives treatment. The backpack and enclosed outer and inner PD fluid supply containers may be removed from the patient and placed next to the patient, e.g., during treatment when the patient has arrived at a destination where the patient needs to be stationary for a period of time. The backpack may further include structure, e.g., one or more hook, for hanging the outer and inner containers, e.g., having one or more aperture for receiving the one or more hook, such that a bottom of the outer supply container is raised up from the bottom of the backpack during operation. In an alternative embodiment, the outer supply container and nested inner container are supported by the bottom of the backpack during operation.

The PD fluid supply assembly may further include a one or more clamp, e.g., a PD fluid supply clamp for clamping the PD fluid supply line and a pneumatic line clamp for clamping the positive pneumatic line leading to the outer supply container. The PD fluid supply clamp and the pneumatic line clamp may for example be fixed to an inner surface of the backpack, wherein the patient when setting up the wearable PD system for use places the PD fluid supply line and the positive pneumatic line into the PD fluid supply clamp and the pneumatic line clamp, respectively, wherein the lines are held fixed to the clamps during treatment and until the patient removes the lines after treatment.

The PD fluid supply assembly may still further include a heater or warmer, e.g., a heating or warming blanket located within the backpack. The heater or warmer is in one embodiment configured to heat itself to patient temperature, e.g., 37 °C, which in the closed environment of the backpack brings the PD fluid to, or close to, patient temperature in an efficient manner and without the possibility of overheating. Similar to the level sensor, the outer supply container may be provided with one or more temperature sensor to contact the inner PD fluid supply container to obtain a temperature of the PD fluid contained within. A simple on/off temperature control may be used such that the heater or warmer is energized until the one or more temperature sensor reads 37 °C, after which the heater or warmer is deenergized until the one or more temperature sensor reads an acceptable lower temperature, e.g., 32 °C, after which the above cycle is repeated.

The pumping unit of the wearable PD system of the present disclosure in one embodiment includes an air or pneumatic pump having a positive pressure outlet and a negative pressure outlet. The pumping unit also includes a control unit, which controls whether the air pump is pumping or not, whether each of the clamps is open or closed, and whether the heater or warmer (if provided) is energized or not. The control unit also receives outputs from any of the sensors associated with the wearable PD system. The pumping unit may be located within the backpack or be worn by the patient elsewhere, such as on the patient's belt.

The control unit of the pumping unit provides or operates with a user interface. For example, if the pumping unit is worn on the patient's belt, then the user interface may be located on the control unit. Here, the user interface may include a touchscreen display and/or one or more button, such as a membrane switch, to allow the patient to enter data and commands into the user interface. If the pumping unit is located instead within the backpack, then the user interface may be provided as a mobile application that is downloaded onto and played via a user's smartphone or tablet (smart device). Here, the control unit includes a transceiver for wireless two-way communication with the user's smartphone or tablet. Upon installation of the mobile application, the application pairs or registers with the control unit of the wearable PD system of the present disclosure, after which the smartphone and the control unit will only communicate with each other. Also, after pairing or registration, the mobile application and the control unit will sync automatically upon power-up of both the smartphone and the control unit.

The user interface allows the patient after setup to start a treatment using the wearable PD system of the present disclosure. The user interface allows the patient to pause or stop treatment at any time. Upon starting treatment, the user interface may display a message to the patient to confirm that each of the PD fluid supply assembly, pumping unit and PD fluid drain assembly have been setup and connected together. Upon receiving confirmation from the patient, the control unit begins treatment and causes automatic operation of each of the air or pneumatic pump, the clamps and the PD fluid heater or warmer if provided.

Regarding pneumatic line communication, the positive pneumatic line leading to the outer supply container is connected to the positive pressure outlet of the air or pneumatic pump. A second, negative pneumatic line is provided and connects to the negative pressure outlet of the air or pneumatic pump. The negative pneumatic line leads to one or more outer leg container of the PD fluid drain assembly. Each one or more outer leg container operates with a nested inner PD fluid drain container. The outer leg container(s) and the inner PD fluid drain container(s) are cylindrical in one embodiment, such that they may slide up over one or both of the patient's feet and come to rest around one or both of the patient's legs. The outer leg container(s) may again be rigid, e.g., a rigid plastic or flexible, e.g., an airtight fabric, such as canvas. If rigid, the outer leg container(s) include an openable and sealable top that allows the inner PD fluid drain container(s) to be placed within the outer leg container(s). The rigid top and a rigid cylindrical body of the outer leg container(s) may be sealed together via inner and outer o-rings and the closure of one or more releasable latch to hold the rigid top and the rigid cylindrical body together in a sealed manner. If flexible, the lid may include an airtight outer zipper and inner o-ring seal for sealing the lid to the inner cylindrical wall of the flexible cylindrical body of the outer leg container(s).

The inner PD fluid drain container(s) are flexible plastic containers in one embodiment. The inner PD fluid drain container(s) are cylindrically shaped and in one embodiment include a drain line welded or otherwise permanently connected to the container so that the drain line cannot be pulled off of the container by accident while the patient walks or is otherwise mobile. The lid of the outer leg container(s) is provided with a stretchable, spandex-like sealing sleeve that allows a connector located on the connecting end of the drain line to be slid through the sealing sleeve, after which the sealing sleeve seals to the drain line.

PD fluid drain assembly also includes a drain clamp for each drain line. The drain clamps are located in one embodiment at the lid and clamp onto the respective drain line just above the sealing sleeve. The patient inserts the drain line into the drain clamp, which releasably holds the drain line during treatment. The lid of each outer leg container is also provided with a pneumatic connector for connecting to the negative pneumatic line extending from the air or pneumatic pump. The lid of each outer leg container is further provided with a pneumatic clamp for clamping the negative pneumatic line.

The PD fluid supply assembly, the pumping unit and the PD fluid drain assembly are each battery powered in one embodiment. The battery power is in one implementation a rechargeable battery power. A separate battery may be provided for each of the PD fluid supply assembly, the pumping unit and the PD fluid drain assembly. Here, electrical wires running to the fluid and pneumatic clamps are not necessary, and the clamps may be actuated wirelessly, e.g., over the same WIFI network that allows the user interface on the user's smartphone to interact with the control unit. The patient may at the end of the day plug a recharging chord into each of the three separate batteries.

In an alternative embodiment, a single battery is provided with the pumping unit. Imbedded and insulated wires are located along an inner surface of the backpack and run to the fresh PD fluid line clamp, the pneumatic line clamp and the heater or warmer if provided. If the pumping unit is located within the backpack, then no electrical connection needs to be made by the patient between the pumping unit and the PD fluid supply assembly. If the pumping unit is located instead along the patient's belt, for example, then the patient makes an electrical connection between the pumping unit and the PD fluid supply assembly. It is also contemplated to locate the negative pneumatic line within an insulating sleeve and to also run electrical lines through the insulating sleeve to the lid of each outer leg container. The electrical lines exit the sleeve at the lid and connect to each of the used PD fluid line clamp and the pneumatic line clamp.

The disposable set of the wearable PD system of the present disclosure includes the nested inner PD fluid supply container, e.g., standard PD fluid container, the PD fluid supply line, one or more PD fluid drain line, one or more PD fluid drain container, and a line connection manifold. The line connection manifold includes a transfer set connector for connecting to the patient's PD fluid transfer set and at least one drain connector for connecting to the one or more PD fluid drain line. The PD fluid supply line extends in one embodiment from the line connection manifold, through an opening in the backpack and to the supply connector located at the nested inner PD fluid supply container. The PD fluid supply line in an alternative embodiment is welded or otherwise permanently fixed to the nested inner PD fluid supply container and connects instead to a supply connector located at the line connection manifold. In any case, the patient makes two and possibly three fluid connections, one for the supply connector and one or two for the one or two drain connectors.

The patient also makes one or two pneumatic connections, one for each negative pneumatic line and pneumatic connector located at one or more outer leg container. After treatment, the patient removes each drain line from the line connection manifold and removes the negative pneumatic line from each outer leg container, so that each outer leg container may be removed from the patient. It is contemplated that the positive pneumatic line remains connected between the pumping unit and the outer supply container of the PD fluid supply assembly, especially if the pumping unit is located within the backpack. If the pumping unit is located instead at the patient's belt, then it is contemplated to allow the patient to disconnect both the PD fluid supply line (which may be manually clamped before removal) and the positive pneumatic line, so that the backpack is then untethered from the patient and may be removed and left behind after the patient fill is complete.

If a heater or warmer is provided, control unit may cause the heater or warmer to be energized with the outer supply container and the PD fluid supply container located within the backpack prior to making treatment connections. If the heater draws too much power from the battery, a separate AC plug may be provided by the backpack for powering the heater or warmer. It is contemplated also to not provide a heater and instead instruct the patient to store the PD fluid supply containers in a warm location.

After the fresh PD fluid is heated, the above-described connections are made, and the patient begins treatment using the user interface, the control unit causes (i) the PD fluid supply clamp to open, (ii) the positive pressure pneumatic clamp to open, and (iii) the air pump to energize and pump positive pressure air to the outer supply container of the PD fluid supply assembly. The positive air pressure may for example be 1.5 psig to 3.0 psig. The control unit also briefly causes one of the drain clamps to open and for the user interface to prompt the patient to look for fresh PD fluid in the line connection manifold, indicating that the PD fluid supply line has been primed. Upon the patient confirming at the user interface that the PD fluid supply line has been primed, the user interface next prompts the patient to open the manual valve of the patient's transfer set. The positive pressure air pressurizes the space between the outer supply container and the nested inner PD fluid supply container, forcing fresh PD fluid to flow from the inner PD fluid supply container, through the PD fluid supply line, through the line connection manifold, through the patient's transfer set and through the patient's indwelling catheter into the patient's peritoneal cavity. In an embodiment, the control unit receives a signal from the level sensor indicating that the PD fluid supply container is empty or almost empty. The control unit may cause the air pump to stop or to wait for a period of time, e.g., one or more minute, before causing the air pump to stop.

In an alternative embodiment, the level sensor is not provided and the control unit instead energizes the air pump for a period of time sufficient to expect that all or virtually all of the fresh PD fluid has been delivered from the inner PD fluid supply container to the patient's peritoneal cavity. It is contemplated to allow the patient to press a stop button at the user interface at any time during the patient fill (likely near the end) when the patient feels full enough. When the patient fill has ended, the control unit removes power from the PD fluid supply clamp and the positive pressure pneumatic clamp, which close in a failsafe manner in one embodiment. The control unit also removes power from the heater, if provided, and if still powered at the end of the patient fill.

After the air pump is stopped, the control unit begins a timed patient dwell. The timed patient dwell may last for one to two hours. At any time while the patient is sitting or otherwise not moving, even during the patient fill, the patient may remove the backpack and set it next to the patient, e.g., at or above the patient's peritoneal cavity so that gravity aids the patient fill. As discussed above, it may be possible to untether the patient from the backpack after the patient fill and during the patient dwell.

When the timed patient dwell ends, as determined by the control unit, the control unit causes (i) the one or more drain clamp to open, (ii) the negative pressure pneumatic clamp to open, and (iii) the air pump to energize and pull negative pressure at the one or more outer leg container. The negative pressure is applied to the one or more PD fluid drain container and is transmitted through the one or more drain line, the line connection manifold, the patient's transfer set and the patient's indwelling catheter to the used PD fluid residing within the patient. The negative pneumatic pressure accordingly helps to begin to cause the used PD fluid to flow from the patient's peritoneal cavity to the one or more PD fluid drain container. In an embodiment, the control unit energizes the air pump for a period of time sufficient to expect that all or virtually all of the used PD fluid has been drained from the patient's peritoneal cavity to the one or more PD fluid drain container. Depending on the size of the PD fluid supply container, e.g., two liters, it may be desirable to provide two PD fluid drain containers, e.g., each holding one liter of used PD fluid plus an expected amount of patient ultrafiltration, so that the weight of used PD fluid per leg is lessened and is distributed evenly.

In light of the disclosure set forth herein, and without limiting the disclosure in any way, in a first aspect of the present disclosure, which may be combined with any other aspect, or portion thereof, a wearable peritoneal dialysis (“PD”) system comprises a PD fluid supply assembly including an outer supply container, and an inner PD fluid supply container sealed within the outer supply container; a pumping unit including an air pump having a positive pressure outlet and a negative pressure outlet; a PD fluid drain assembly including at least one outer leg container configured to be worn around a patient's leg, and at least one inner PD fluid drain container sealed within the at least one outer leg container; and a control unit configured to cause (i) positive pneumatic pressure to be supplied from the positive pressure outlet to the outer supply container to force fresh PD fluid to flow from the inner PD fluid supply container to the patient and (ii) negative pneumatic pressure to be supplied from the negative pressure outlet to the at least one inner container to force used PD fluid to flow from the patient towards the inner PD fluid drain container.

In a second aspect of the present disclosure, which may be combined with any other aspect, or portion thereof, the control unit is configured to cause (i) the positive pneumatic pressure to be supplied during a patient fill and (ii) negative pneumatic pressure to be supplied during a patient drain.

In a third aspect of the present disclosure, which may be combined with any other aspect, or portion thereof, the control unit is located within the pumping unit.

In a fourth aspect of the present disclosure, which may be combined with any other aspect, or portion thereof, the outer supply container is configured to open and close about the inner PD fluid supply container, and wherein the outer supply container includes a sealed interface for sealing the PD fluid supply container within the outer supply container.

In a fifth aspect of the present disclosure, which may be combined with any other aspect, or portion thereof, a supply connector extends from the inner PD fluid supply container through the outer supply container in a sealed manner, and wherein a PD fluid supply line extends from the supply connector towards a PD fluid transfer set carried by the patient.

In a sixth aspect of the present disclosure, which may be combined with any other aspect, or portion thereof, the wearable PD system includes a PD fluid supply clamp under control of the control unit, and wherein the PD fluid supply line extends through the PD fluid supply clamp.

In a seventh aspect of the present disclosure, which may be combined with any other aspect, or portion thereof, the wearable PD system includes a line connection manifold in fluid communication with the PD fluid transfer set, and wherein the PD fluid supply line extends to the line connection manifold.

In an eighth aspect of the present disclosure, which may be combined with any other aspect, or portion thereof, the at least one drain line extends from the at least one inner PD fluid drain container in a sealed manner through the at least one outer leg container and towards a PD fluid transfer set carried by the patient.

In a ninth aspect of the present disclosure, which may be combined with any other aspect, or portion thereof, the wearable PD system includes at least one drain clamp under control of the control unit, and wherein the at least one drain line extends through the at least one drain clamp.

In a tenth aspect of the present disclosure, which may be combined with any other aspect, or portion thereof, the wearable PD system includes a line connection manifold in fluid communication with the PD fluid transfer set, and wherein the at least one drain line extends to the line connection manifold.

In an eleventh aspect of the present disclosure, which may be combined with any other aspect, or portion thereof, the at least one drain line extends from the at least one inner PD fluid drain container in a sealed manner through a stretchable sealing sleeve of the at least one outer leg container.

In a twelfth aspect of the present disclosure, which may be combined with any other aspect, or portion thereof, the wearable PD system includes a positive pneumatic line extending from the positive pressure outlet to the outer supply container.

In a thirteenth aspect of the present disclosure, which may be combined with any other aspect, or portion thereof, the wearable PD system includes a pneumatic line clamp under control of the control unit, and wherein the positive pneumatic line extends through the pneumatic line clamp.

In a fourteenth aspect of the present disclosure, which may be combined with any other aspect, or portion thereof, the wearable PD system includes at least one negative pneumatic line extending from the negative pressure outlet to the at least one outer leg container.

In a fifteenth aspect of the present disclosure, which may be combined with any other aspect, or portion thereof, the wearable PD system includes at least one pneumatic line clamp under control of the control unit, and wherein the at least one negative pneumatic line extends through the at least one pneumatic line clamp.

In a sixteenth aspect of the present disclosure, which may be combined with any other aspect, or portion thereof, the at least one negative pneumatic line extends to a sealable lid of the at least one outer leg container.

In a seventeenth aspect of the present disclosure, which may be combined with any other aspect, or portion thereof, the at least one negative pneumatic line extends through at least one insulating sleeve along with an electrical wire to the at least one outer leg container.

In an eighteenth aspect of the present disclosure, which may be combined with any other aspect, or portion thereof, the outer supply container, the inner PD fluid supply container and optionally (i) the pumping unit and (ii) the control unit are located within a backpack wearable by the patient.

In a nineteenth aspect of the present disclosure, which may be combined with any other aspect, or portion thereof, the wearable PD system includes a heater or warmer provided within the backpack, the heater or warmer positioned and arranged to heat fresh PD fluid located within the inner PD fluid supply container.

In a twentieth aspect of the present disclosure, which may be combined with any other aspect, or portion thereof, the wearable PD system of Claim 1, which includes a mobile application provided on a smart device in data communication with the control unit, the mobile application providing a user interface for the wearable PD system.

In a twenty-first aspect of the present disclosure, which may be combined with any other aspect, or portion thereof, a wearable peritoneal dialysis (“PD”) system comprises a PD fluid supply assembly including an outer supply container, and an inner PD fluid supply container sealed within the outer supply container; an air pump; a control unit configured to cause positive pneumatic pressure to be supplied from the air pump to the outer supply container to force fresh PD fluid to flow from the inner PD fluid supply container to a patient; and a backpack, wherein the outer supply container, the inner PD fluid supply container and optionally the air pump and the control unit are located within the backpack.

In a twenty-second aspect of the present disclosure, which may be combined with any other aspect, or portion thereof, the wearable PD system includes at least one outer leg container configured to be worn around a patient's leg and at least one inner PD fluid drain container sealed within the at least one outer leg container, wherein the air pump includes a positive pressure outlet and a negative pressure outlet, and wherein the control unit is configured to cause (i) the positive pneumatic pressure to be supplied from the positive pressure outlet to the outer supply container to force fresh PD fluid to flow from the inner PD fluid supply container to the patient and (ii) the negative pneumatic pressure to be supplied from the negative pressure outlet to the at least one inner container to force used PD fluid to flow from the patient towards the inner at least one PD fluid drain container.

In a twenty-third aspect of the present disclosure, which may be combined with any other aspect, or portion thereof, any of the features, functionality and alternatives described in connection with any one or more of FIGS. 1 and 2 may be combined with any of the features, functionality and alternatives described in connection with any other of FIGS. 1 and 2.

In light of the present disclosure and the above aspects, it is therefore an advantage of the present disclosure to provide a wearable PD system that is relatively simple and inexpensive.

It is another advantage of the present disclosure to provide a PD treatment that may be performed while the patient is mobile.

It is yet another advantage of the present disclosure to provide a wearable PD system that is at least partially removable when the patient is not moving.

It is still another advantage of the present disclosure to provide a wearable PD system that is at least partially removable after a patient fill phase has been completed.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a diagram of one embodiment for a wearable peritoneal dialysis system of the present disclosure.

FIG. 2 is a side elevation view of a patient wearing the wearable peritoneal dialysis system of the present disclosure.

### DETAILED DESCRIPTION

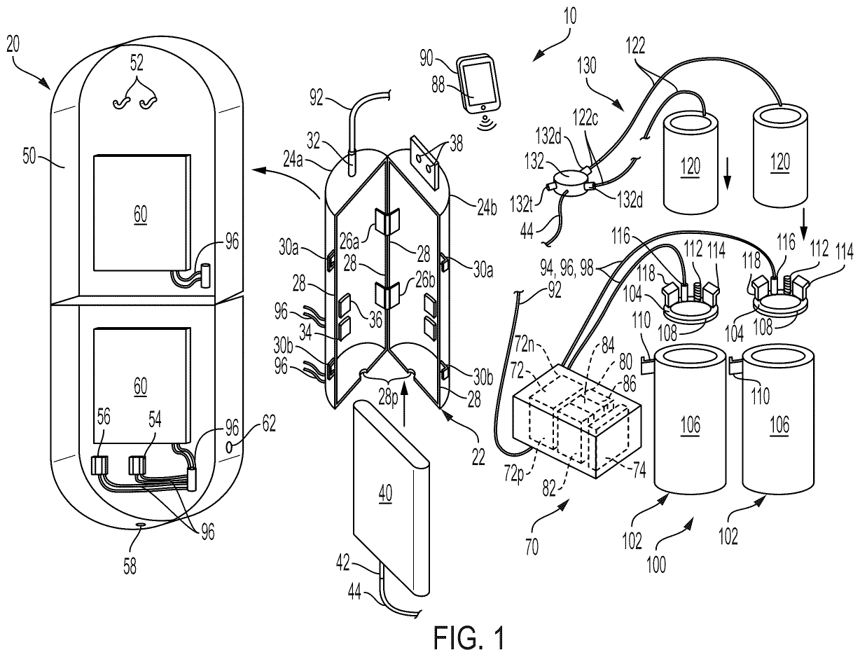

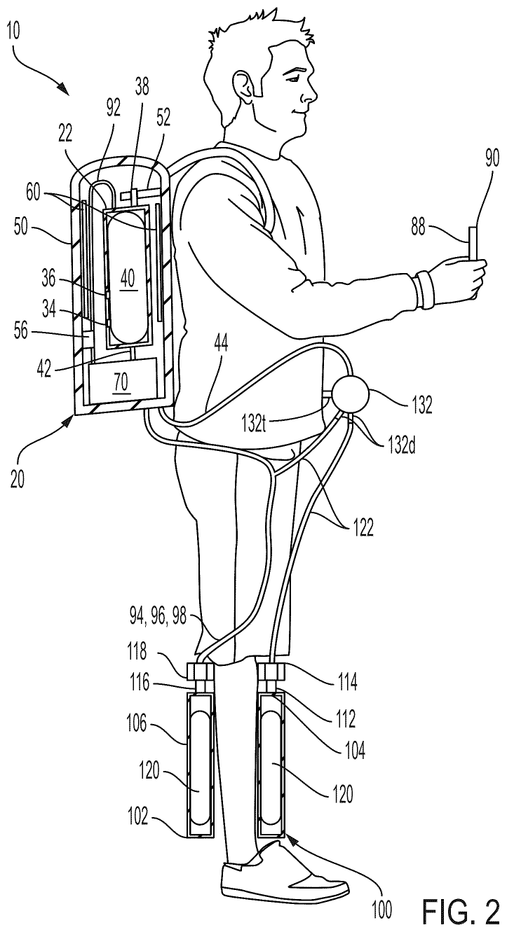

Example systems, methods, and apparatus are disclosed herein for a wearable peritoneal dialysis (“PD”) system 10 that performs a PD treatment while the patient is either moving or in a situation requiring the patient to have to move. Referring now to FIGS. 1 and 2, example PD system 10 in one embodiment includes three primary components, namely, a PD fluid supply assembly 20, a pumping unit 70 and a PD fluid drain assembly 100. PD fluid supply assembly 20 includes an outer supply container 22 and an inner PD fluid supply container 40. Outer supply container 22 may be rigid, such as a rigid plastic, or be a flexible container, such as a flexible airtight fabric, e.g., canvas, which does not stretch substantially when placed under positive pneumatic pressure. Outer supply container 22 in one embodiment includes first and second halves 24a, 24b that are openable, e.g., via one or more hinge 26a, 26b, so as to be able to receive a new inner PD fluid supply container, which may be a standard presterilized PD fluid bag. Outer supply container 22 includes a sealed interface 28, e.g., via a compressible gasket, such that when outer supply container 22 is closed about the inner PD fluid supply container, the outer supply container seals in an airtight manner about the inner PD fluid supply container. Outer supply container 22 may include one or more latch 30a, 30b that releasably close to compress sealed interface 28 during operation of wearable PD system 10 of the present disclosure. If outer supply container 22 is flexible, e.g., canvas, sealed interface 28 may include an airtight zipper. Regardless of the material for outer supply container 22, sealed interface 28 in an embodiment also includes a portion 28p that seals around a supply connector 42, e.g., standard PD fluid bag connector, which extends from the inner PD fluid supply container. In this way, supply connector 42 may be connected to a PD fluid supply line 44 outside of outer supply container 22. In a similar manner, outer supply container 22 includes a pneumatic connector 32 that may be accessed and connected to a positive pneumatic line 92 outside of outer supply container 22.

Outer supply container 22 is reusable and in one embodiment includes one or more level sensor 34 located at a bottom of the container for sensing when the PD fluid located within inner PD fluid supply container 40 is almost complexly gone, having been delivered to the patient. Sensor 34 may be any type of sensor that can detect, through a wall of the inner PD fluid supply container, whether PD fluid or air is present. Sensor 34 may for example be an ultrasonic sensor, magnetic sensor, capacitive sensor or inductive sensor. Outer supply container 22 and inner PD fluid supply container 40 are here sized such that at their bottoms, inner PD fluid supply container 40 is held snugly within the outer supply container 22 so that sensor 34 is consistently pressed up against a wall of inner fluid supply container 40 to provide an accurate output signal.

PD fluid supply assembly 20 in one embodiment further includes a backpack 50 worn by the patient and into which outer supply container 22 and nested inner PD fluid supply container 40 are placed while the patient moves from one location to another for whatever reason, and while the patient receives treatment. Backpack 50 and enclosed outer and inner PD fluid supply containers 22, 40 may be removed from the patient and placed next to the patient, e.g., during treatment when the patient has arrived at a destination where the patient needs to be stationary for a period of time. Backpack 50 may further include structure, e.g., one or more hook 52, for hanging the outer and inner containers, e.g., having one or more aperture 38 for receiving the one or more hook, such that a bottom of outer supply container 22 is raised up from the bottom of the backpack 50 during operation. In an alternative embodiment, outer supply container 22 and nested inner container 40 are supported by the bottom of the backpack 50 during operation.

PD fluid supply assembly 20 may further include a one or more clamp 54, e.g., a PD fluid supply clamp for clamping PD fluid supply line 44 and a pneumatic line clamp 56 for clamping positive pneumatic line 92 leading to outer supply container 22. PD fluid supply clamp 54 and pneumatic line clamp 56 may for example be fixed to an inner surface of the backpack 50, wherein the patient when setting up wearable PD system 10 for use places PD fluid supply line 44 and positive pneumatic line 92 into PD fluid supply clamp 54 and pneumatic line clamp 56, respectively, wherein lines 44, 92 are held fixed to clamps 54, 56 during treatment and until the patient removes the lines after treatment.

PD fluid supply assembly 20 may still further include a heater or warmer 60, e.g., a heating or warming blanket located within backpack 50. Heater or warmer 60 is in one embodiment configured to heat itself to patient temperature, e.g., 37 °C, which in the closed environment of backpack 50 brings the PD fluid to, or close to, patient temperature in an efficient manner and without the possibility of overheating. Similar to the level sensor, outer supply container 22 may be provided with one or more temperature sensor 36 to contact inner PD fluid supply container 40 to obtain a temperature of the PD fluid contained within. A simple on/off temperature control may be used such that the heater or warmer 60 is energized until the one or more temperature sensor 36 reads 37 °C, after which the heater or warmer is deenergized until the one or more temperature sensor 36 reads an acceptable lower temperature, e.g., 32 °C, after which the above cycle is repeated.

Pumping unit 70 of wearable PD system 10 of the present disclosure in one embodiment includes an air or pneumatic pump 72 having a positive pressure outlet 72p and a negative pressure outlet 72n. Suitable air pumps for air pump 72 include those found in portable blood pressure machines, which may be battery powered. Pumping unit 70 also includes a control unit 80, which includes one or more processor 82, one or more memory 84 and possibly a video controller 86 for displaying information on a user interface 88. Control unit 80 controls whether the air pump 72 is pumping or not, whether each of the clamps 54, 56, 114, 118 is open or closed, and whether heater or warmer 60 (if provided) is energized or not. Control unit 80 also receives outputs from any of the sensors 34, 36 associated with wearable PD system 10. Pumping unit 70 may be located within backpack 50 or be worn by the patient elsewhere, such as on the patient's belt.

As mentioned above, control unit 80 of pumping unit 70 provides or operates with a user interface 88. For example, if pumping unit 70 is worn on the patient's belt, then user interface 80 may be located on control unit 80. Here, user interface 88 may include a touchscreen display and/or one or more button, such as a membrane switch, to allow the patient to enter data and commands into the user interface. If pumping unit 70 is located instead within backpack 50, then user interface 88 may be provided as a mobile application that is downloaded onto and played via a user's smartphone or tablet (smart device) 90. Here, control unit 80 further includes a transceiver for wireless two-way communication with the user's smartphone or tablet 90. Upon installation of the mobile application, the application pairs or registers with control unit 80 of wearable PD system 10 of the present disclosure, after which smartphone 90 and control unit 80 will only communicate with each other. Also, after pairing or registration, the mobile application and control unit 80 will sync automatically upon power-up of both smartphone 90 and control unit 80.

User interface 88 allows the patient after setup to start a treatment using wearable PD system 10 of the present disclosure. User interface 88 allows the patient to pause or stop treatment at any time. Upon starting treatment, user interface 88 may display a message to the patient to confirm that each of PD fluid supply assembly 20, pumping unit 70 and PD fluid drain assembly 100 have been setup and connected together. Upon receiving confirmation from the patient, control unit 80 begins treatment and causes automatic operation of each of the air or pneumatic pump 72, the clamps 54, 56, 114, 118 and PD fluid heater 60 or warmer if provided.

Regarding pneumatic line communication, positive pneumatic line 92 leading to outer supply container 22 is connected to positive pressure outlet 72p of the air or pneumatic pump 72. A second, negative pneumatic line 94 is provided and connects to negative pressure outlet 72n of air or pneumatic pump 72. Negative pneumatic line 94 leads to one or more outer leg container 102 of PD fluid drain assembly 100. Each one or more outer leg container 102 operates with a nested inner PD fluid drain container 120. Outer leg container(s) 102 and the inner PD fluid drain container(s) 120 are cylindrical in one embodiment, such that they may slide up over one or both of the patient's feet and come to rest around one or both of the patient's legs. Outer leg container(s) 102 may again be rigid, e.g., a rigid plastic or flexible, e.g., an airtight fabric, such as canvas (or canvas having an inner rubber lining). If rigid, outer leg container(s) 102 include an openable and sealable top 104 that allows the inner PD fluid drain container(s) 120 to be placed within outer leg container(s) 102. Rigid top 104 and a cylindrical body 106 of outer leg container(s) 102 may be sealed together via inner and outer o-rings 108 and the closure of one or more releasable latch 110 to hold rigid top 104 and cylindrical body 106 together in a sealed manner. If flexible, the lid 104 may include an airtight outer zipper and inner o-ring seal for sealing the lid to the inner cylindrical wall of cylindrical body 106 of outer leg container(s) 102. Also, if made to be flexible, outer leg container(s) 102 may each include one or more flexible hook and loop type fastener for fastening the outer leg container(s) to the patient's leg(s).

Inner PD fluid drain container(s) 120 are flexible plastic containers in one embodiment. Inner PD fluid drain container(s) 120 are also cylindrically shaped and in one embodiment include a drain line 122 welded or otherwise permanently connected to container 120 so that drain line 122 cannot be pulled off of the container by accident while the patient walks or is otherwise mobile. Lid 104 of outer leg container(s) 102 is provided with a stretchable, spandex-like sealing sleeve 112 that allows a connector 122c located on the connecting end of drain line 122 to be slid through sealing sleeve 112, after which the sealing sleeve seals to drain line 122.

PD fluid drain assembly 100 also includes a drain clamp 114 for each drain line 122. Drain clamps 114 are located in one embodiment at lid 104 and clamp onto a respective drain line 122 just above sealing sleeve 112. The patient inserts drain line 122 into drain clamp 114, which releasably holds drain line 122 during treatment. Lid 104 of each outer leg container 102 is also provided with a pneumatic connector 116 for connecting to negative pneumatic line 94 extending from air or pneumatic pump 72. Lid 104 of each outer leg container 102 is further provided with a pneumatic clamp 118 for clamping negative pneumatic line 94.

PD fluid supply assembly 20, pumping unit 70 and PD fluid drain assembly 100 are each battery powered in one embodiment. The battery power is in one implementation a rechargeable battery power. A separate battery may be provided for each of the PD fluid supply assembly 20, pumping unit 70 and PD fluid drain assembly 100. Here, electrical wires running to the fluid and pneumatic clamps are not necessary, and the clamps may be actuated wirelessly, e.g., over the same WIFI network that allows user interface 88 on the patient's smartphone or tablet (smart device) 90 to interact with control unit 80. The patient may at the end of the day plug a recharging chord into each of the three separate batteries.

In the illustrated embodiment, a single battery 74 is provided with pumping unit 70. Imbedded and insulated electrical wires or wires 96 are located along an inner surface of backpack 50 and run to fresh PD fluid line clamp 54, pneumatic line clamp 56 and heater or warmer 60 if provided. If pumping unit 70 is located within the backpack 50, then no electrical connection (for wires 96) needs to be made by the patient between pumping unit 70 and PD fluid supply assembly 20. If pumping unit 70 is located instead along the patient's belt, for example, then the patient makes an electrical connection (for wires 96) between pumping unit 70 and PD fluid supply assembly 20. It is also contemplated to locate negative pneumatic line 94 within an insulating sleeve 98 and to also run electrical lines 96 through the insulating sleeve to lid 104 of each outer leg container 102. Electrical lines 96 exit sleeve 98 at lid 104 and connect to each of drain clamp 114 and pneumatic line clamp 118. If control unit 70 is located within backpack 50, then negative pneumatic line 94, electrical lines 96 and sleeve 98 may exit backpack to reach fluid drain assembly 100 via an aperture 62 provided in the backpack as illustrated in FIG. 1.

A disposable set 130 of wearable PD system 10 of the present disclosure includes nested inner PD fluid supply container 40, e.g., standard PD fluid container, PD fluid supply line 44, one or more PD fluid drain line 122, one or more PD fluid drain container 120, and a line connection manifold 132. Line connection manifold 132 includes a transfer set connector 132t for connecting to the patient's PD fluid transfer set and at least one drain connector 132d for connecting to one or more PD fluid drain line 122. PD fluid supply line 44 extends in one embodiment from line connection manifold 132, through an opening 58 in backpack 50 and to supply connector 42 located at nested inner PD fluid supply container 40. PD fluid supply line 44 in an alternative embodiment is welded or otherwise permanently fixed to nested inner PD fluid supply container 40 and connects instead to a supply connector (not illustrated) located at the line connection manifold 132. In any case, the patient makes two and possibly three fluid connections, one for supply connector 42 and one or two for the one or two drain connectors 132d of line connection manifold 132.

The patient also makes one or two pneumatic connections, one for each negative pneumatic line 94 and pneumatic connector 116 located at one or more outer leg container 102. After treatment, the patient removes each drain line 122 from line connection manifold 132 and removes negative pneumatic line 94 from each outer leg container 102, so that each outer leg container 102 may be removed from the patient. It is contemplated that positive pneumatic line 92 remains connected between pumping unit 70 and outer supply container 22 of PD fluid supply assembly 20, especially if pumping unit 70 is located within backpack 50. If pumping unit 70 is located instead at the patient's belt, then it is contemplated to allow the patient to disconnect both PD fluid supply line 44 (which may be manually clamped before removal) and positive pneumatic line 92, so that backpack 50 is then untethered from the patient and may be removed and left behind after the patient fill is complete.

Any rigid component of system 10, such as outer supply container 22 (in one embodiment), outer leg container 102 (in one embodiment), the enclosure for pumping unit 70 and line connection manifold 132 may be made of plastic, such as, polyvinyl chloride (“PVC”), polyethylene (“PE”), polyurethane (“PU”) or polycarbonate (“PC”) or of metal, such as stainless steel. Any flexible components of system 10, such as PD fluid supply container 40, one or more PD fluid drain container 120, PD fluid supply line 44, one or more drain line 122, positive pneumatic line 92, and negative pneumatic line 94, may be made of a medically safe material such as one or more plastic, e.g., PVC, PE, PU, or other suitable non-PVC polymer.

If heater or warmer 60 is provided, control unit 80 may cause the heater or warmer to be energized with outer supply container 22 and PD fluid supply container 40 located within backpack 50 prior to making treatment connections. If heater 60 draws too much power from the battery 74, a separate AC plug (not illustrated) may be provided by backpack 50 for powering heater or warmer 60. It is contemplated also to not provide a heater 60 and instead instruct the patient to store PD fluid supply containers 40 in a warm location.

After the fresh PD fluid is heated, the above-described connections are made, and the patient begins treatment using user interface 88, control unit 80 causes (i) PD fluid supply clamp 54 to open, (ii) positive pressure pneumatic clamp 56 to open, and (iii) air pump 72 to energize and pump positive pressure air to outer supply container 22 of PD fluid supply assembly 20. The positive air pressure may for example be 1.5 psig to 3.0 psig. Control unit 80 also briefly causes one of drain clamps 114 to open and for user interface 88 to prompt the patient to look for fresh PD fluid in line connection manifold 132, indicating that PD fluid supply line 44 has been primed. Upon the patient confirming at the user interface that PD fluid supply line 44 has been primed, user interface 88 next prompts the patient to open the manual valve of the patient's transfer set. The positive pressure air pressurizes the space between outer supply container 22 and nested inner PD fluid supply container 40, forcing fresh PD fluid to flow from inner PD fluid supply container 22, through PD fluid supply line 44, through line connection manifold 132, through the patient's PD fluid transfer set and through the patient's indwelling catheter into the patient's peritoneal cavity. In an embodiment, control unit 80 receives a signal from level sensor 34 indicating that PD fluid supply container 40 is empty or almost empty. Control unit 80 may cause air pump 72 to stop or to wait for a period of time to ensure container 40 is empty, e.g., one or more minute, before causing air pump 72 to stop.

In an alternative embodiment, level sensor 34 is not provided and control unit 80 instead energizes air pump 72 for a period of time sufficient to expect that all or virtually all of the fresh PD fluid has been delivered from inner PD fluid supply container 40 to the patient's peritoneal cavity. It is contemplated to allow the patient to press a stop button at user interface 88 at any time during the patient fill (likely near the end) when the patient feels full enough. When the patient fill has ended, control unit 80 removes power from PD fluid supply clamp 54 and positive pressure pneumatic clamp 56, which close in a failsafe manner in one embodiment. Control unit 80 also removes power from heater 60, if provided, and if still powered at the end of the patient fill.

After air pump 72 is stopped, control unit 80 begins a timed patient dwell. The timed patient dwell may last for one to two hours for example. At any time while the patient is sitting or otherwise not moving, even during the patient fill, the patient may remove backpack 50 and set it next to the patient, e.g., at or above the patient's peritoneal cavity so that gravity aids the patient fill. As discussed above, it may be possible to untether the patient from backpack 50 after the patient fill and during the patient dwell.

When the timed patient dwell ends, as determined by control unit 80, the control unit causes (i) the one or more drain clamp 114 to open, (ii) negative pressure pneumatic clamp 118 to open, and (iii) air pump 72 to energize and pull negative pressure at one or more outer leg container 102. The negative pressure is applied to one or more PD fluid drain container 120 and is transmitted through one or more drain line 122, line connection manifold 132, the patient's transfer set and the patient's indwelling catheter to the used PD fluid residing within the patient. The negative pneumatic pressure accordingly helps to begin to cause the used PD fluid to flow from the patient's peritoneal cavity to one or more PD fluid drain container 120, which flow is aided by gravity. In an embodiment, control unit 80 energizes air pump 72 for a period of time sufficient to expect that all or virtually all of the used PD fluid has been drained from the patient's peritoneal cavity to one or more PD fluid drain container 120. It may be found that once a steady stream of used PD fluid is flowing to drain container(s) 120 due to gravity that air pump 72 may be deenergized. Depending on the size of PD fluid supply container 40, e.g., two liters, it may be desirable to provide two PD fluid drain containers 120, e.g., each holding one liter of used PD fluid plus an expected amount of patient ultrafiltration, so that the weight of used PD fluid per leg is lessened and is distributed more evenly.

FIG. 2 shows a patient wearing one embodiment of wearable PD system 10 of the present disclosure. The patient wears backpack 50, which is part of PD fluid supply assembly 20. Backpack 50 stores outer supply container 22 and nested inner PD fluid supply container 40, wherein outer supply container 22 includes a bracket defining one or more aperture 38, which is/are hung onto hooks 52 extending from an inner surface of backpack 50. Pumping unit 70, including air pump 72, control unit 80 and battery 74, is therefore able to be located beneath outer supply container 22 and nested inner PD fluid supply container 40. PD fluid supply line 44, connected to supply connector 42 of supply container 40, is also able to extend downwardly from the supply container and out through an aperture defined in backpack 50, which is desirable because air will migrate to the top of the supply container and gravity will help feed fresh PD fluid from the supply container to the patient's peritoneal cavity.

Heater or warmer 60 is provided and located on the inner walls of backpack 50. Heater 60 may be started, e.g., via user interface 88 provided on smart device 90, prior to the patient donning backpack 50 and becoming mobile. Heater may be powered via an AC plug (not illustrated), which also recharges battery 74. Outer supply container 22 includes one or more level sensor 34 for detecting when the level of fresh PD fluid within supply container 40 falls to an empty or almost empty condition. Outer supply container 22 also includes one or more temperature sensor 36 for detecting the temperature of fresh PD fluid within supply container 40, e.g., for use during heating.

Positive pneumatic line 92 extends from the negative pressure outlet 72n of air pump 72 and through pneumatic line clamp 56. When the patient presses a “start fill” button on user interface 88 of smart device 90, control unit 80 causes pneumatic line clamp 56 to energize open and air pump 72 to deliver positive air pressure to the space between outer supply container 22 and nested inner PD fluid supply container 40, pushing fresh PD fluid from the PD fluid supply container. Control unit 80 causes PD fluid supply clamp 54 (not viewable in FIG. 2) to energize open to allow fresh PD fluid to flow through PD fluid supply line 44.

PD fluid supply line 44 extends to line connection manifold 132, which is located adjacent to the patient's PD fluid transfer set. Line connection manifold 132 includes a transfer set connector 132t for connecting to the patient's transfer set and at least one drain connector 132d for connecting to at least one drain line 122. Two drain lines 122 are shown in FIG. 2. One drain line 122 extends to nested inner PD fluid drain container 120 (as seen in FIG. 2), while the other drain line 122 extends to a nested inner PD fluid drain container 120 located on the patient's other leg, which is not seen in FIG. 2. As illustrated in FIG. 2, drain line 122 extends through stretchable, spandex-like sealing sleeve 112 to cylindrical PD fluid drain container 120. Sealing sleeve 112 is located on a top or lid 104 of outer leg container 102, wherein top or lid 104 is removably sealed in an airtight manner to cylindrical body 106 of the outer leg container.

Pneumatic connector 116, drain clamp 114 and pneumatic clamp 118 are also provided or supported by top or lid 104 of outer leg container 102. Drain clamp 114 is configured to clamp drain line 122 in a failsafe manner (same for all clamps herein in one embodiment). When control unit 80 in one embodiment initiates a patient drain, drain clamp 114 is caused to open to allow used PD fluid to flow through drain line 122 into PD fluid drain container 120.

Pneumatic clamp 118 is configured to clamp pneumatic line 94 (located within insulating sleeve 98 along with electrical lines 96 leading to the clamps) in a failsafe manner. When control unit 80 initiates the patient drain, pneumatic clamp 118 is caused to open to allow negative pressure from negative pressure outlet 72n of air pump 72 to begin to draw used PD fluid to flow from the patient into drain line 122, where gravity then aids in the flow of the used PD fluid into PD fluid drain container 120.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A wearable peritoneal dialysis (“PD”) system comprising:
a PD fluid supply assembly including
an outer supply container, and
an inner PD fluid supply container sealed within the outer supply container;
a pumping unit including an air pump having a positive pressure outlet and a negative pressure outlet;
a PD fluid drain assembly including
at least one outer leg container configured to be worn around a patient's leg, and
at least one inner PD fluid drain container sealed within the at least one outer leg container; and
a control unit configured to cause (i) positive pneumatic pressure to be supplied from the positive pressure outlet to the outer supply container to force fresh PD fluid to flow from the inner PD fluid supply container to the patient and (ii) negative pneumatic pressure to be supplied from the negative pressure outlet to the at least one inner container to force used PD fluid to flow from the patient towards the inner PD fluid drain container.

2. The wearable PD system of claim 1, wherein the control unit is configured to cause (i) the positive pneumatic pressure to be supplied during a patient fill and (ii) negative pneumatic pressure to be supplied during a patient drain.

3. The wearable PD system of claim 1, wherein the control unit is located within the pumping unit.

4. The wearable PD system of claim 1, wherein the outer supply container is configured to open and close about the inner PD fluid supply container, and wherein the outer supply container includes a sealed interface for sealing the PD fluid supply container within the outer supply container.

5. The wearable PD system of claim 1, wherein a supply connector extends from the inner PD fluid supply container through the outer supply container in a sealed manner, and wherein a PD fluid supply line extends from the supply connector towards a PD fluid transfer set carried by the patient.

6. The wearable PD system of claim 5, which includes a PD fluid supply clamp under control of the control unit, and wherein the PD fluid supply line extends through the PD fluid supply clamp.

7. The wearable PD system of claim 5, which includes a line connection manifold in fluid communication with the PD fluid transfer set, and wherein the PD fluid supply line extends to the line connection manifold.

8. The wearable PD system of claim 1, wherein the at least one drain line extends from the at least one inner PD fluid drain container in a sealed manner through the at least one outer leg container and towards a PD fluid transfer set carried by the patient.

9. The wearable PD system of claim 8, which includes at least one drain clamp under control of the control unit, and wherein the at least one drain line extends through the at least one drain clamp.

10. The wearable PD system of claim 8, which includes a line connection manifold in fluid communication with the PD fluid transfer set, and wherein the at least one drain line extends to the line connection manifold.

11. The wearable PD system of claim 8, wherein the at least one drain line extends from the at least one inner PD fluid drain container in a sealed manner through a stretchable sealing sleeve of the at least one outer leg container.

12. The wearable PD system of claim 1, which includes a positive pneumatic line extending from the positive pressure outlet to the outer supply container.

13. The wearable PD system of claim 12, which includes a pneumatic line clamp under control of the control unit, and wherein the positive pneumatic line extends through the pneumatic line clamp.

14. The wearable PD system of claim 1, which includes at least one negative pneumatic line extending from the negative pressure outlet to the at least one outer leg container.

15. The wearable PD system of claim 14, which includes at least one pneumatic line clamp under control of the control unit, and wherein the at least one negative pneumatic line extends through the at least one pneumatic line clamp.

16. The wearable PD system of claim 14, wherein the at least one negative pneumatic line extends to a sealable lid of the at least one outer leg container.

17. The wearable PD system of claim 14, wherein the at least one negative pneumatic line extends through at least one insulating sleeve along with an electrical wire to the at least one outer leg container.

18. The wearable PD system of claim 1, wherein the outer supply container, the inner PD fluid supply container and optionally (i) the pumping unit and (ii) the control unit are located within a backpack wearable by the patient.

19. The wearable PD system of claim 18, which includes a heater or warmer provided within the backpack, the heater or warmer positioned and arranged to heat fresh PD fluid located within the inner PD fluid supply container.

20. The wearable PD system of claim 1, which includes a mobile application provided on a smart device in data communication with the control unit, the mobile application providing a user interface for the wearable PD system.

21. A wearable peritoneal dialysis (“PD”) system comprising:
a PD fluid supply assembly including
an outer supply container, and
an inner PD fluid supply container sealed within the outer supply container;
an air pump;
a control unit configured to cause positive pneumatic pressure to be supplied from the air pump to the outer supply container to force fresh PD fluid to flow from the inner PD fluid supply container to a patient; and
a backpack, wherein the outer supply container, the inner PD fluid supply container and optionally the air pump and the control unit are located within the backpack.

22. The wearable PD system of claim 21, which includes at least one outer leg container configured to be worn around a patient's leg and at least one inner PD fluid drain container sealed within the at least one outer leg container, wherein the air pump includes a positive pressure outlet and a negative pressure outlet, and wherein the control unit is configured to cause (i) the positive pneumatic pressure to be supplied from the positive pressure outlet to the outer supply container to force fresh PD fluid to flow from the inner PD fluid supply container to the patient and (ii) the negative pneumatic pressure to be supplied from the negative pressure outlet to the at least one inner container to force used PD fluid to flow from the patient towards the inner at least one PD fluid drain container.
